(19) United States
(12) Patent Application Publication
(10) Pub. No.: US 2017/0319192 A1
(43) Pub. Date: Nov. 9, 2017

(54) SUTURE ANCHORS HAVING RIBBED ENHANCEMENTS

(71) Applicant: Smith & Nephew, Inc., Memphis, TN (US)

(72) Inventors: Mark R. Guy, Memphis, TN (US); Mark Edwin Housman, Memphis, TN (US); Geoffrey Ian Karasic, Memphis, TN (US); Nehal Patel, Memphis, TN (US)

(73) Assignee: Smith & Nephew, Inc., Memphis, TN (US)

(21) Appl. No.: 15/526,233

(22) PCT Filed: Dec. 11, 2015
(86) PCT No.: PCT/US2015/065212

§ 371 Date: May 11, 2017

Related U.S. Application Data
(60) Provisional application No. 62/090,676, filed on Dec. 11, 2014.
(60) Provisional application No. 62/090,662, filed on Dec. 11, 2014.
(60) Provisional application No. 62/090,652, filed on Dec. 11, 2014.

(51) Int. Cl. A61B 17/04

(52) U.S. Cl. CPC A61B 17/0401; A61B 2017/0414; A61B 2017/0403; A61B 2017/0409; A61B 2017/0412

(57) ABSTRACT

A knotless interference or contact fixation anchor assembly comprising an anchor body with a removable inserter/driver that fully supports the suture anchor over its entire working length. The anchor body may also incorporating a suture trap that is housed within a proximal eyelet of the anchor body. The anchor body may include a plurality of elongate ribs extending longitudinally along at least a portion of the anchor body. The plurality of ribs comprise flexible barbs. When inserted into bone or a bone tunnel, the barbs compress slightly, resulting in less bone compression around the anchor due to the forces required for insertion, and increased pull-out resistance.

### BACKGROUND

Suture anchors are often used in surgical procedures. For example, a suture anchor may be used for securing soft tissue to bone by means of a suture attached to the suture anchor which is then inserted into the bone. A typical suture anchor is inserted into bone by pounding the suture anchor either into a bone tunnel or directly into the bone. Alternatively, the suture anchor can be configured for insertion by a screw mechanism. The suture anchor may be made of metal, plastic or bioreabsorbable material (which dissolves in the body over time). The suture anchor can include an eyelet that allows the suture to pass therethrough and link the suture anchor and the suture. Upon implantation into bone, the suture anchor engages the bone and resists further movement, providing an anchor point for the attached suture.

In recent years, surgeons have been moving towards the use of smaller suture anchors in surgical repair operations. The use of smaller suture anchors may be less invasive, require less bone removal or disruption at the site of implantation, and allow for more rapid patient healing. In such cases, it is desirable for suture anchors to be as small as possible, without compromising anchor integrity or fixation strength. For “pound-in” type anchors, as the size of the anchor is reduced, the volume of material making up the anchor is also reduced, which can present challenges to the integrity of the anchor during its insertion into bone. In addition, reducing anchor size may present challenges in terms of suture retention within the anchor body, and thus the reliability of the repair. It is therefore desirable that both the integrity of the suture anchor and the reliability of suture retention during insertion is not compromised with the use of smaller suture anchors.

Another desirable feature of suture anchors is increased fixation strength. The fixation strength of a suture anchor to bone is determined by the area of contact between the bone and the suture anchor and the normal force present there between (i.e., frictional sliding resistance). Assuming a constant normal force, as the contact area is increased, the fixation strength generally increases and vice versa. With the user of smaller suture anchors, however, less surface area is available for frictional engagement with the surrounding bone. Thus, lower fixation strength is observed in smaller suture anchors. As a consequence, such suture anchors may not be suitable for certain repair operations, where a certain level of fixation may be required.

### SUMMARY

Described herein are “pound-in” suture anchors that preserve and/or increase fixation strength with bone when implanted. Embodiments include knotless interference or contact fixation anchors that includes an anchor body having a plurality of external ribs orientated with the longitudinal axis of the anchor body. Multiple cuts are provided in one or more of the side walls, edges or corners of the ribs, creating flexible barbs. When inserted into bone or a bone tunnel, the barbs compress slightly. Advantageously, this results in less bone compression around the suture anchor due to the slightly reduced forces required for insertion, in comparison to anchor which does not include such barbs, making for an easier insertion and promotion of boney ingrowth and healing. In addition, the suture anchor described herein is more resistant to removal as the barbs engage with the inner wall of the bone tunnel and flex as the anchor is pulled proximally. As a result, fixation strength is enhanced as the collective resistance of the multiple barbs provide an appreciable gain in pull-out resistance. An additional advantage is that, in an uncompressed state, the suture anchor will have a slightly larger diameter than the bone tunnel, thereby permitting tunnels of smaller diameters to be used or, in the alternative, smaller diameter anchors.

Embodiments of the suture anchor described herein also include a pound-in suture anchor with a removable inserter/driver that fully supports the suture anchor over its entire working length. The removable inserter advantageously provides support over the length of the suture anchor during installation and can be removed from the suture anchor once the suture or sutures have been loaded.

Embodiments of the suture anchor described herein also include a pound-in suture anchor incorporating a suture trap that is housed within a proximal eyelet of the suture anchor. In a pre-implanted state, the suture trap is located at the distal end of the eyelet. When a force is applied, either by contact with bone during implantation, or removal of the metal inserter, the suture trap is driven toward the proximal end of the eyelet. Advantageously, the suture is then impinged within the eyelet by cooperating locking mechanisms on the suture trap and within the eyelet.

In various embodiments of the suture anchor described herein, the suture anchor may include an elongated anchor body having a proximal end and a distal end, and a longitudinal axis extending between the proximal and distal ends and a plurality of elongate ribs extending longitudinally along at least a portion of the anchor body, with at least one of the plurality of ribs including at least one compressible barb. The anchor body may further include a transverse bore and a pair of channels extending proximally from the transverse bore, the channels suitable for receiving suture or tape. The anchor may include a tapered tip formed at the distal end of the anchor body. The distal end of the tapered tip may have a conical shape, and may be rounded or pointed.

In further embodiments, at least one the plurality of ribs extends from the proximal end of the anchor body to a position proximal to the distal end or to a selection position within the tip. At least one of the plurality of ribs may include a plurality of barbs. At least a portion of the plurality of elongate ribs may be tapered. Each of the plurality of ribs may include at least one barb or a plurality of barbs. Each of the plurality of ribs may include a pair of sidewalls, with the at least one barb or plurality of barbs located in the sidewalls. Each of the plurality of ribs may include a pair of sidewalls and a top surface, with the at least one barb or plurality of barbs located in the sidewalls, the top surface, or both the sidewalls and the top surface. A portion of the plurality of elongate ribs may be tapered.

In still further embodiments, a length of the at least one compressible barb or plurality of barbs may be uniform or vary along the length of the rib. The at least one barb or plurality of barbs may be located in an edge of the ribs, between the sidewalls or between the sidewalls and the top surface. The at least one barb or plurality of barbs may comprise a first material selected from the group including poly(lactic-co-glycolic) acid (PLGA), β-Tricalcium phosphate (β-TCP) and calcium sulfate, poly-L-lactic acid-hydroxyapatite (PLLA-HA), poly-D-lactide (PDLA), polyether ether ketone (PEEK) or variants thereof, and bioabsorbable materials.

In still further embodiments, the suture anchor may comprise a tapered tip formed at the distal end of the anchor body. The tapered tip may comprise a distal tip end formed from a second material that is harder than the first material. The second material has a hardness within the range between about 40 Shore D to about 85 Shore D. The suture anchor according to claim 12, wherein at least one of the plurality of ribs extends from the proximal end of the anchor body to a position proximal to the distal tip end or to a selection position within the tapered tip. The anchor body may further comprise an opening transverse to the longitudinal axis configured for the passage of a suture. A suture trap may be housed within the opening and configured to impinge a suture within the opening.

In still further embodiments, a suture anchor assembly may include a suture anchor having an elongated anchor body having a proximal end and a distal end, and a longitudinal axis extending between the proximal and distal ends, and a plurality of elongate ribs extending longitudinally along at least a portion of the anchor body, at least one of the plurality of ribs comprising at least one compressible barb, and an inserter removably coupled to the suture anchor body. The inserter may further comprise a slot defined by first and second prongs, the slot in communication with an opening transverse to the longitudinal axis of the anchor body and configured for the passage of a suture, the slot allowing for passage of the suture between the first and second prongs when the inserter is removed from the anchor body.

### BRIEF DESCRIPTION OF THE DRAWINGS

The foregoing and other objects, features and advantages will be apparent from the following more particular description of the examples, as illustrated in the accompanying drawings in which like reference characters refer to the same parts throughout the different views. The drawings are not necessarily to scale, emphasis instead being placed upon illustrating the principles of the examples.

FIG. 1 is an illustration of an embodiment of the suture anchor assembly of the present disclosure in an assembled state;

FIGS. 2a-b are illustrations of another embodiment of the suture anchor assembly in a disassembled state;

FIGS. 3-4 are cross-sectional illustrations of other embodiments of the suture anchor assembly; and

FIG. 5 is an illustration of a section view of an embodiment of the suture anchor assembly

FIG. 6 is an illustration of an embodiment of the suture anchor of the present disclosure in a pre-loaded, pre-inserted state;

FIG. 7 is a cross-section of the embodiment of FIG. 6 assembled with the suture anchor;

FIG. 8 is an illustration of another embodiment of the suture anchor in a post-inserted state;

FIGS. 9a-d are schematic illustrations of embodiments of the suture trap of the present disclosure;

FIGS. 10a and 10b are schematic illustrations of alternative embodiments of the suture trap of the present disclosure;

FIG. 11 is close-up isometric partial view of the ribs of the embodiment of FIG. 1 barbs shown; and

FIGS. 12-18 are illustrations of further examples of the barbs of FIG. 11.

### DETAILED DESCRIPTION

In the description that follows, like components have been given the same reference numerals, regardless of whether they are shown in different examples. To illustrate an example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

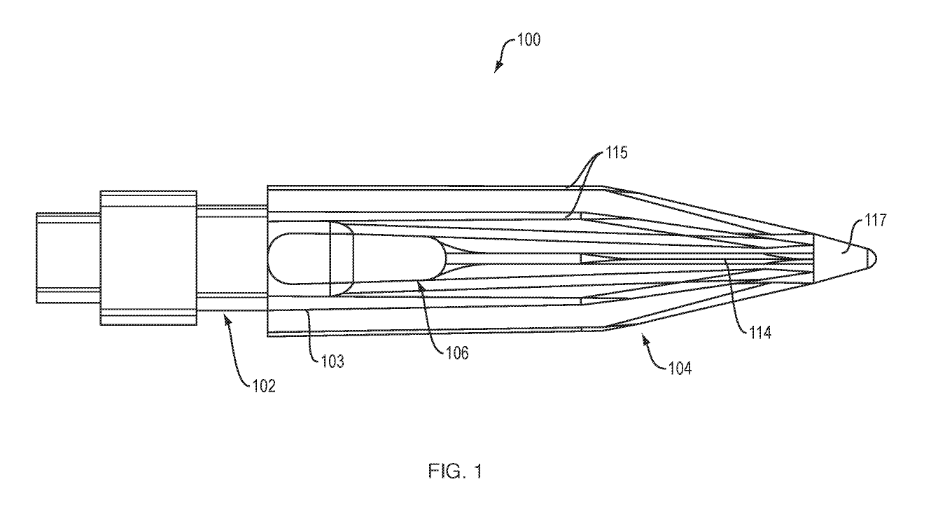

Referring now to FIG. 1, an embodiment of the suture anchor assembly 100 of this disclosure having full anchor axial support is shown in a transparent view and in an assembled state. The suture anchor assembly 100 may be generally comprised of an inserter 102 and an anchor body 104. The anchor body 104 is shown in FIG. 1 as a tapered anchor body 104, but other anchors (including non-tapered anchors) are also possible. The anchor body 104 may be tubular, possessing a circular or elliptical cross-section. In alternative examples, the cross-section of the anchor body 104 may adopt different closed shapes. The anchor body 104 may include a tapered tip 114 with a distal tip end 117. An eyelet 106 may be located at the proximal end of the anchor body 104. It may be noted that, because of the body taper as shown in the anchor body 104 of FIG. 1, inclusion of distal eyelet of sufficient size to receive sutures is not feasible. However, because of the wider body shown at the proximal end of the anchor body 104, a sufficiently large eyelet 106 can be placed to accept large suture loads which may be doubled-over in combination with a passer. The anchor body 104 may also include an internal cannulation 116. The cannulation 116 is primarily for receiving part of the inserter 102 so that the anchor body 104 may be mounted on the inserter 102 prior to its insertion in bone. This arrangement also permits the anchor body 104 to be delivered more precisely, and helps to prevent rotational movement of the anchor body 104 as it is inserted into bone.

The anchor body 104 may be partially or entirely formed from a first material selected from a formulation of poly(lactic-co-glycolic) acid (PLGA), β-Tricalcium phosphate (β-TCP) and calcium sulfate, poly-L-lactic acid-hydroxyapatite (PLLA-HA), poly-D-lactide (PDLA), polyether ether ketone (PEEK) or variants thereof. Biocomposite examples of the anchor body 104 made from a combination of PLGA, β-TCP, and calcium sulfate are absorbable by the body, which is beneficial to natural healing. An example formulation of PLGA, β-TCP, and calcium sulfate is described in U.S. Pat. No. 8,545,866, the entirety of which is herein incorporated by reference. A copolymer of polyglycolic acid (PGA) and polytrimethylene carbonate (TMC) is another example of a bioreabsorbable material. Other commonly used materials for implants are also contemplated by this disclosure. In any case, the anchor body 104 comprises a material that is capable of providing the strength needed to set the fixation device into position and to hold the suture and tissue in position while bone-to-tissue in-growth occurs.

The distal tip end 117 may have a conical, round, pointed or other suitable shape. The distal tip end 117 may be formed from a second material, different from the first material. The second material is harder than the first material, reflecting the fact that the distal tip end 117 is responsible for displacing a majority of the bone volume occupied by the anchor body 104, including both the hard, outer cortical bone layer and the underlying cancellous bone. For example, the distal tip end 117 may be formed from a material having a hardness within the range between about 40 Shore D to about 85 Shore D. In further embodiments, examples of the second material may include, but are not limited to, stainless steels, titanium, titanium alloys, cobalt-chromium alloys, platinum alloys, and palladium alloys, carbon-reinforced polyether ether ketone (PEEK), and glass-reinforced PEEK.

The anchor body 104 also includes a plurality of longitudinal ribs 115 extending from the distal tip end 117 of the anchor body 104 to the proximal end of the anchor body 104. The plurality of ribs 115 are formed on the outer surface of the suture anchor body 104, circumferentially spaced and extending radially outward therefrom. The plurality of ribs 115 are generally elongate and extend longitudinally along the anchor body 104. In the example of FIG. 1, the plurality of ribs 115 extends substantially from the proximal end of the anchor body 104 to a position proximal to the distal end or to a selected point within the tip 114. Furthermore, the portion of the ribs 115 formed within the tip 114 are configured to follow the taper of the tip 114. In alternative embodiments, not shown, the ribs 115 formed with the tip 114 are not aligned with the ribs 115 of the anchor body 104, and the tip 114 may comprise a greater number of ribs 115 than the anchor body 104, or fewer ribs, some of which may or may not be aligned with the ribs 115 of the anchor body 104.

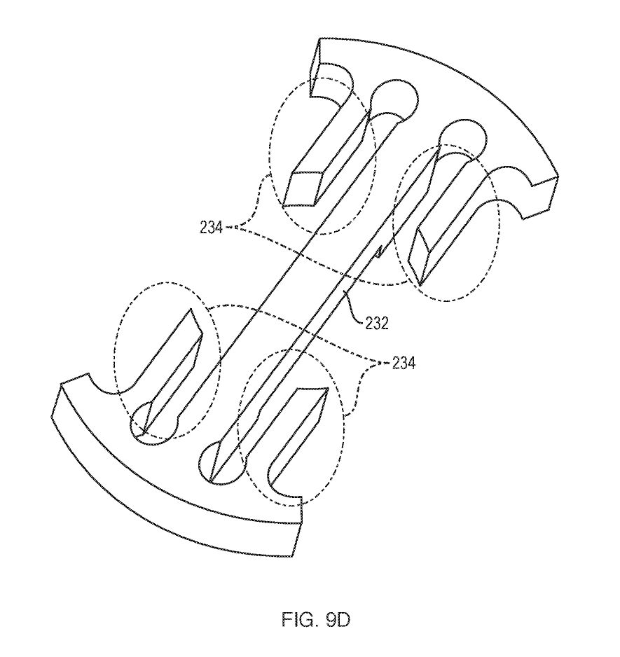

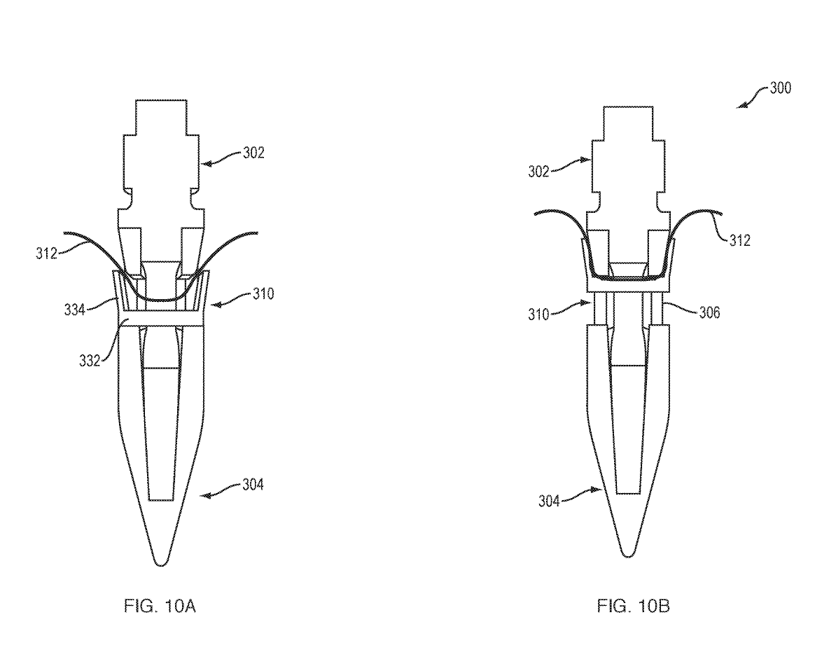

In further alternative examples, not shown, the plurality of ribs 115 may be include breaks along their length, and the anchor body 104 may comprise both ribs 115 which include breaks along their length and ribs 115 without such breaks. In further examples, each of the plurality of ribs 115 may be straight-sided, without any curved portions. For example, in FIG. 1, the cross-sectional shape of the plurality of ribs 115 is shown as generally rectangular. In alternative embodiments, not shown, the ribs 115 have a triangular or rounded cross-section. Each of the plurality of ribs 115 may be laterally offset or spaced from one another by a distance, which may be same distance around the circumference of the anchor body 104 or may vary. Additional embodiments of the plurality of ribs 115 are shown with reference to FIGS. 10-17 as further described below.

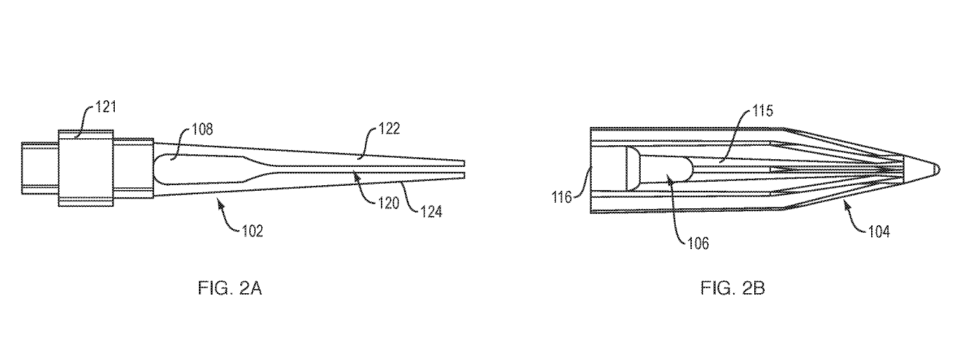

FIG. 2a shows the inserter 102 disassembled from the anchor body 104 (FIG. 2b). The inserter 102 may comprise a main body 121 including first and second prongs 122, 124 extending from the main body 121 and capable of insertion into the anchor body 104 through the cannulation 116 in the anchor body 104, thus providing structural support for the entire length of the anchor body 104. The inserter 102 may also comprise an opening 118 which may at least partially line up with the eyelet 106 when the inserter 102 is inserted into the anchor body 104. The size of the opening 118 is selected to allow for a large number of sutures to be loaded simultaneously while doubled over using a suture passer. Once the sutures (not shown) have been loaded into the anchor body 104 and the anchor body 104 has been inserted into bone, the inserter 102 can be removed because the loaded sutures move through a suture exit slot 120 in communication with the opening 118 and defined by first and second prongs 122, 124. In FIG. 2a, the suture exit slot 120 is shown as tapering distally from the opening 118 but other configurations are possible. Because the sutures have already been passed through the anchor eyelet 106 and tensioned prior to removal of the inserter 102, the sutures can align and funnel in the suture exit slot 120 with relative ease.

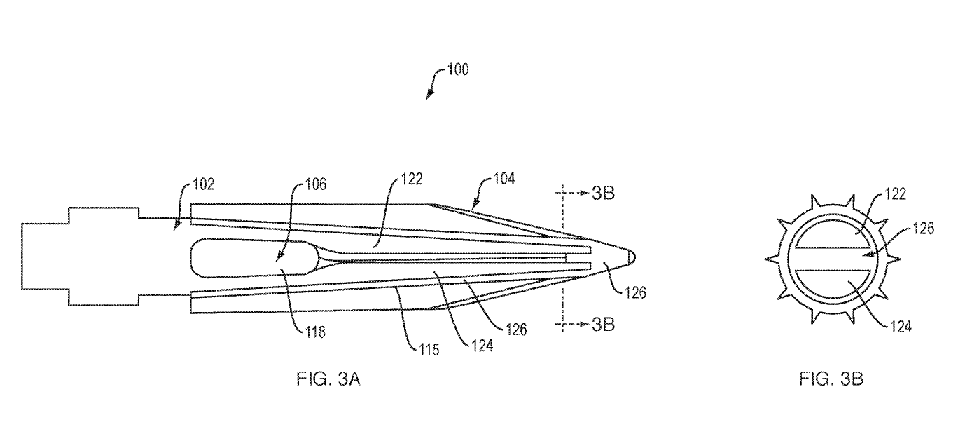

FIG. 3 shows a cross-section of another embodiment of the suture anchor assembly 100, perpendicular to the anchor eyelet 106. The distal end of the anchor body 104 may comprise a slot spacer 126 extending proximally into the cannulation 116 in a variety of lengths. In order to properly orient the inserter 102 to the anchor body 104 both radially and circumferentially, the slot spacer 126 may be configured to mate to the first and second prongs 122, 124 of the inserter 102. The slot spacer 126 may also ensure that the opening 118 aligns with the anchor eyelet 106 when the inserter 102 is inserted into the anchor body 104. The slot spacer 126 may further ensure that first and second prongs 122, 124 of the inserter 102 remain “expanded” circumferentially, thereby ensuring intimate contact between the inserter 102 and the anchor body 104. Intimate contact between the inserter 102 and the anchor body 104 promotes the structural integrity of the suture anchor assembly 100, particularly during the insertion process.

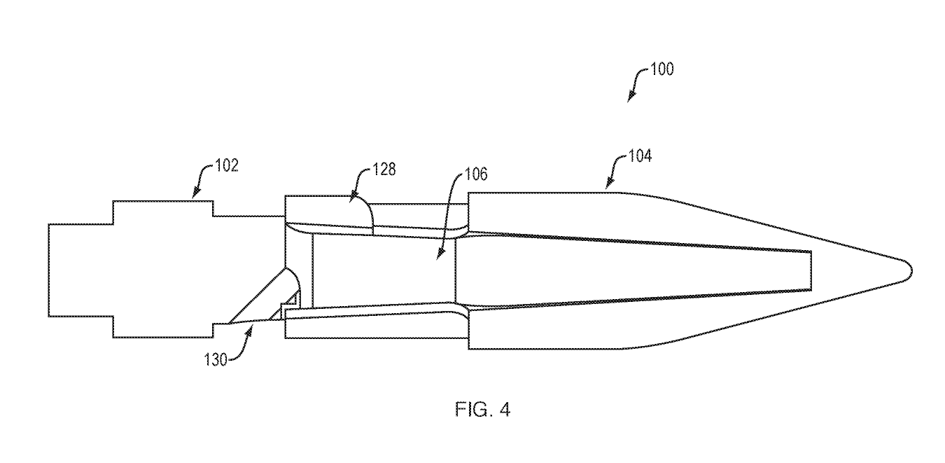

FIG. 4 shows a cross-section of another embodiment of the suture anchor assembly 100 cut through the anchor eyelet 106. In this view, two possible additional features of the suture anchor assembly 100 are shown: a suture bridge 128 and a suture feed slot 130. The suture bridge 128 may be a portion of the anchor body 104 that is sufficiently thick for sutures to bear upon without “cheese wiring” the anchor body 104 when the sutures are pulled on post-implantation. The suture bridge 128 may also serve to impinge sutures against surrounding bone, fixing or locking them in place. The suture bridge 128 may be present on one or both sides of the anchor body 104. The suture feed slot 130 may allow sutures to be tensioned and provide unimpeded ingress into the anchor body 104 during the final stages of inserting the suture anchor assembly 100 into bone.

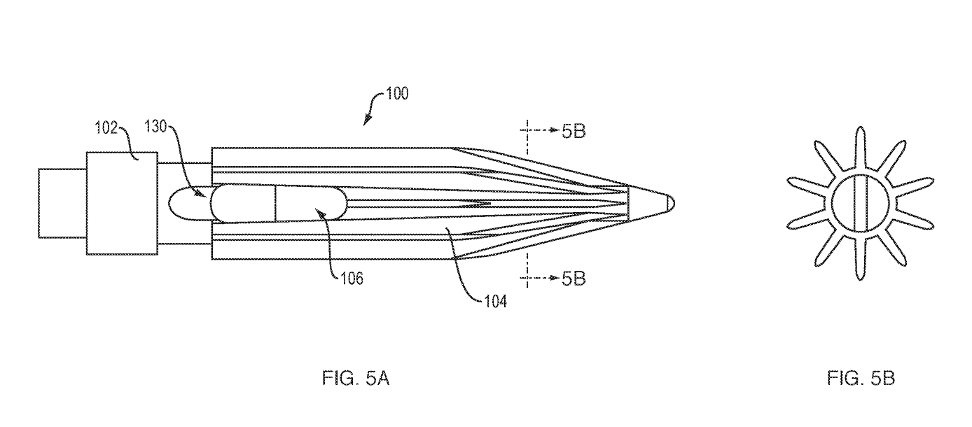

FIG. 5 illustrates an example of how sutures may be loaded into the suture anchor assembly 100. FIG. 5 shows the side of the suture anchor assembly 100 that may contain a suture feed slot 130, 180 degrees opposite from the suture bridge 128 (FIG. 4). FIG. 5 also depicts a typical cross-section of the distal portion of the suture anchor assembly 100. As stated above, in order for the suture to properly feed into the anchor body 104 during pound-in insertion, a suture feed slot 130 may be present in the anchor body 104. During use, the suture bridge side of the anchor body 104 (not shown) may be the side through which the sutures enter the suture anchor assembly 100. The side of the suture anchor assembly 100 containing the suture feed slot 130 may be the side of the suture anchor assembly 100 from which the sutures exit. The suture feed slot 130 may allow the sutures to be better tensioned during surgery and allow the free end of the sutures to flow into the suture anchor assembly 100 when the proximal end of the inserter 102 is pounded into bone.

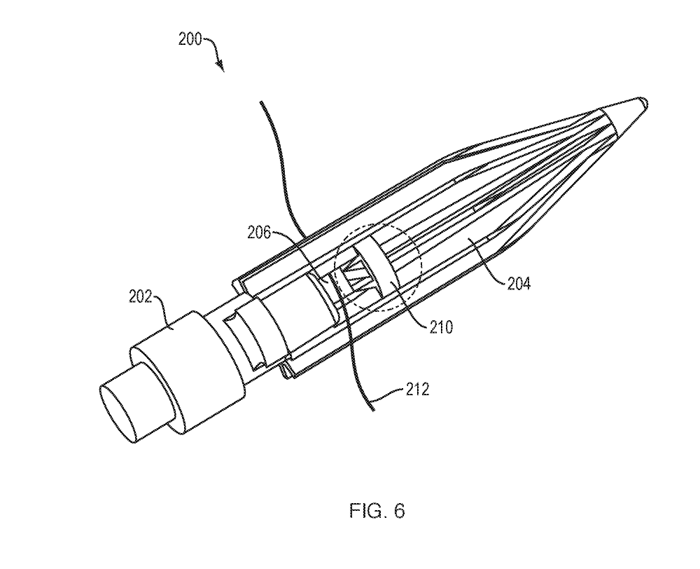

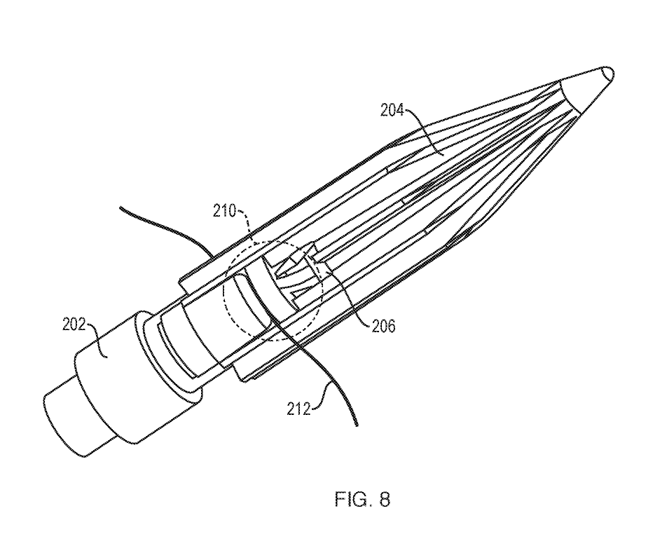

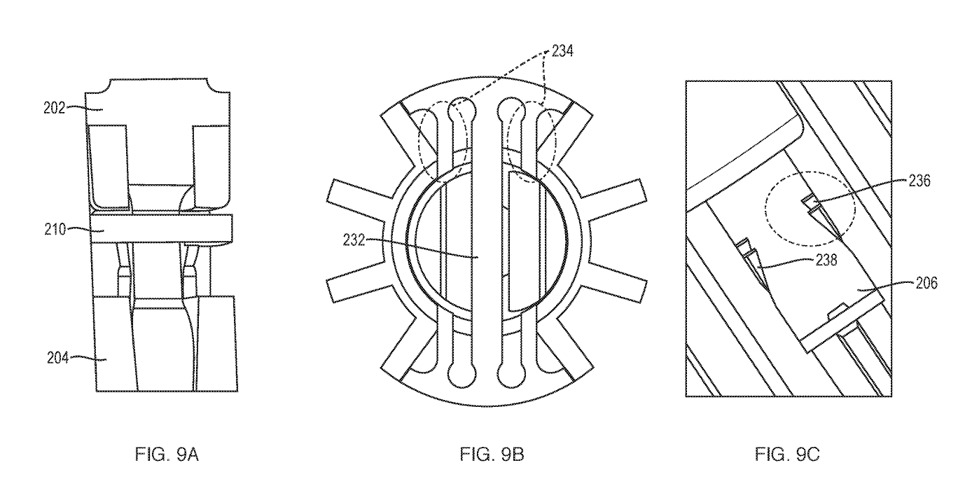

Referring now to FIG. 6, another embodiment of the suture anchor assembly 200 of this disclosure, having a suture capture mechanism, is shown. In FIG. 6, the suture anchor 204 is shown as assembled with inserter 202. An eyelet 206 is located at the proximal end of the suture anchor 200 and transverse to the longitudinal axis of the suture anchor 200. A suture trap 210 (described in greater detail with reference to FIGS. 9a-c) is located at the distal end of eyelet 206. The suture trap 210 may be fabricated out of materials such as PEEK, PLLA-HA, implantable metals, bioreabsorbable or other suitable materials. As shown in FIG. 6, one or more sutures 212 may be passed through the proximal, non-occluded portion of the eyelet 206 with a standard suture passer or by other means. In this state, the one or more sutures 212 are proximal to the suture trap 210.

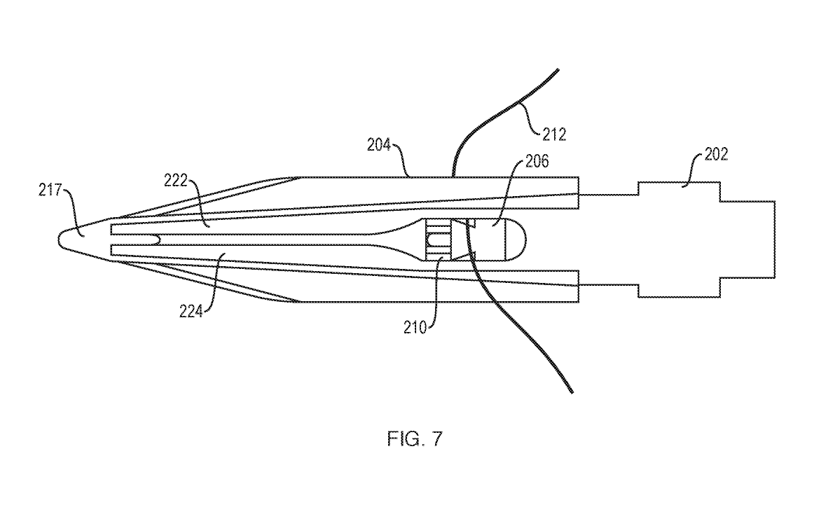

FIG. 7 shows a cross-section of the inserter 202 assembled with the suture anchor 204 of FIG. 6 with suture trap 210 located at the distal portion of the eyelet 206 and the sutures 212 passed therethrough. In use, with the one or more sutures 212 passed through the eyelet 206 of the suture anchor 204, the suture anchor 204 is partially inserted into bone (not shown). With the distal tip end 217 embedded in bone, the sutures 212 passing through the suture anchor 204 are tensioned.

Turning now to FIG. 8, with the sutures properly tensioned, the suture anchor 204 is fully pounded into the bone. Since a portion of the suture trap 210 extends outside of eyelet 206, as the exposed portion of the suture trap 210 encounters the bone surface, the suture trap 210 is driven toward the proximal end of eyelet 206 where it encounters the suture 212 that was passed though the eyelet 206. Once the suture anchor 204 is fully counter-sunk in the bone, inserter 202 can be removed. When inserter 202 is removed, the prongs 222, 224 (FIG. 7) of inserter 202 drag over the suture trap 210, further impinging the suture trap 210 against the suture 212 at the proximal end of the eyelet 206. This mode of action is particularly useful in softer bone situations where impact between bone and the exposed portion of the suture trap 210 is not sufficient to generate full seating of the suture trap 210 at its proximal travel limit. Alternatively, pre-tensioning of suture 212 prior to final insertion of the suture anchor 204 could be used to seat the suture trap 210 proximally if the suture 212 were routed through or around suture trap 212. It is also contemplated by this disclosure that the most lateral aspects of the suture trap 210 could be made to fold onto the suture 212, impinging the suture 212 between the suture anchor 204 and the suture trap 210 after the suture trap 210 is inserted into bone.

FIGS. 9a-c illustrate embodiments of the suture trap 210 of this disclosure in greater detail. FIG. 9a shows a cross-section of the interface of the suture trap 210 with the suture anchor 204 and inserter 202. As shown in FIGS. 9b-c, the suture trap 210 can include a middle spar 232 and four or more flexible elements 234 configured to engage with teeth 236 on a pair of racks 238 formed on an inner surface of the eyelet 206. As the suture trap 210 moves proximally, it rides up over the teeth 236 located in the eyelet 206. The flexible elements 234 and racks 238 (FIG. 9c) cause the suture trap 210 to remain parked distally in the eyelet 206 prior to installation of the suture anchor 204. After installation of the suture anchor 204, the flexible elements 234 and racks 238 cause the suture trap 210 to remain locked proximally in the eyelet 206. This proximally locked position allows for suture impingement between the suture trap 210 and the proximal end of the eyelet 206 to be maintained. The flexible elements 234 and middle spar 232 are shown in more detail in FIG. 9d.

Another embodiment of the suture anchor assembly 300 of this disclosure having a suture capture mechanism is shown in cross-section in FIGS. 10a and 10b. The suture anchor assembly 300 is shown in both a pre-clamped (FIG. 10a) and post-clamped (FIG. 10b) state. The suture anchor assembly 300 generally includes an anchor body 304 and an inserter 302. The anchor body 304 has a proximal end, a tapered distal end, and a longitudinal axis extending between the proximal and distal ends. An eyelet 306 is formed in the proximal end of the anchor body 304, transverse to the longitudinal axis. A suture trap 310 is formed within the eyelet 306 and has at least one flexible member 334, such as wings or flaps, extending radially from the eyelet 306. The suture trap 310 may be fabricated out of materials such as PEEK, PLLA-HA, implantable metals, bioreabsorbable or other suitable materials.

In use, a suture 312 is first routed through the proximal, non-occluded portion of the eyelet 306 (FIG. 10a) with a standard suture passer. In the original, pre-clamping state, the suture 312 is proximal to the suture trap 310. With the suture 312 passed through the eyelet 306, the anchor body 304 is partially hammered into bone (not shown) and the suture 312 is tensioned. Once the suture 312 is tensioned, the anchor body 304 is fully pounded into bone. As the flexible members 334 of the suture trap 310 encounters bone surface of sufficiently hard bone, the suture trap 310 is driven toward the proximal end of the anchor body 304 where it encounters the suture 312 (FIG. 10b) that was passed through the eyelet 306. The inserter 302 is then removed. Removal of the inserter 302 causes a distal portion of the inserter 302 to drag over the middle spar 332 of the suture trap 310, further locking the suture trap 310 against the suture 312 at the proximal end of the eyelet 306. This mode of action is particularly useful in softer bone where impact between bone and the flexible members 334 of the suture trap 310 is not sufficient to generate full seating of the suture trap 310 at its proximal travel limit. Once the suture trap 310 is fully seated and locked, the suture 312 is trapped between the suture trap 310 and the proximal end of the eyelet 306, as well as between the anchor body 304 and surrounding bone.

It is contemplated by this disclosure that flexures and rack teeth, such as those shown in FIGS. 9a-c, could be employed between the suture trap 310 and the eyelet 306. The flexible members 334 could be orientated at a different angle so as to occlude less of the eyelet 306. Pre-tensioning of the suture 312 prior to final insertion of the anchor body 304 could be used to seat the suture trap 310 proximally if the suture 312 were routed through or around the suture trap 310.

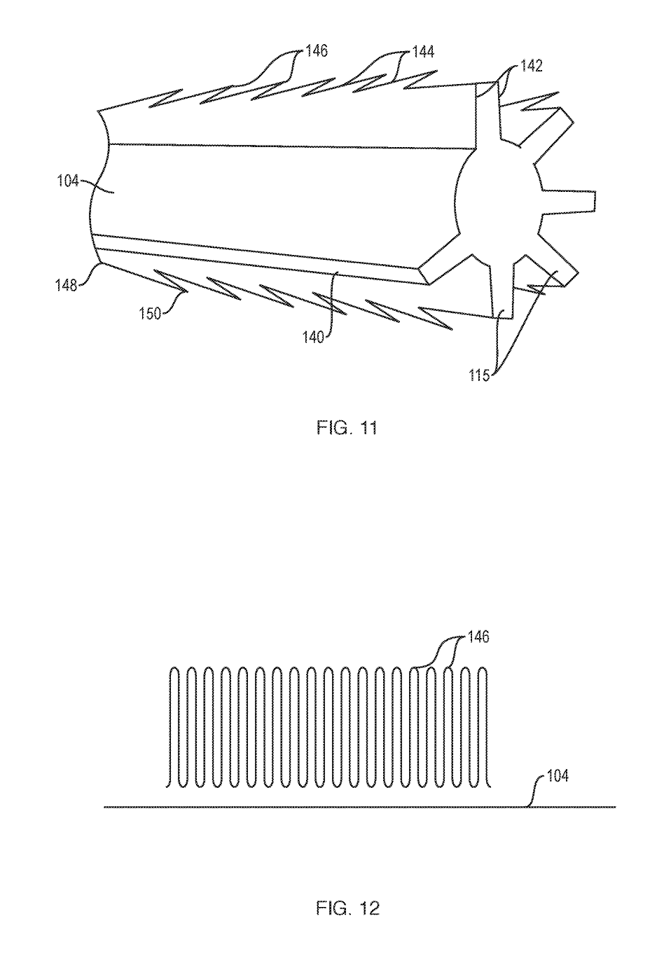

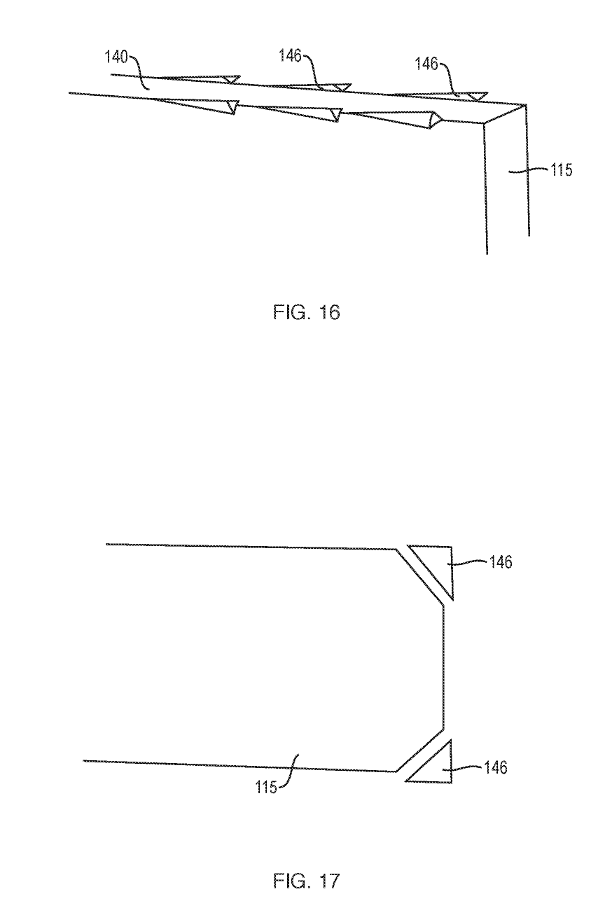

Referring now to FIG. 11, a partial view of a series of the plurality of anchor ribs 115, such as those described with reference to FIG. 1, is illustrated. Various other features of the anchor body 104, both optional and required, have been omitted from this and the subsequent figures for clarity. In FIG. 11, the plurality of ribs 115 are shown as rectangular with a top surface 140 and opposing sidewalls 142. One or more cuts, slashes, slices or ingressions 144 may be made in the plurality of ribs 115 to provide at least one thin, flexible barb 146 formed integrally with the ribs 115, therefore comprising the same material as the rib 115. The barbs 146 may be overlapping, as shown in FIG. 10, or may form non-overlapping units (see FIGS. 15 and 16). The barbs 146 may be formed in a process once the anchor body 104 has been formed, for example by a series of cuts with a laser, blade or hot wire. Alternatively, the barbs 146 can be formed in the anchor molding process. The barbs 146 may comprise a hinge 148 closer to the distal end of the anchor body 104 and a free (cantilevered) end 150 closer to the proximal end of the anchor body 104. In alternative embodiments (see FIG. 12), the barbs 146 are formed perpendicular to the anchor body 104, like the teeth of a comb. The barbs 146 may be present in all of the ribs 115 or in just some of the ribs 115.

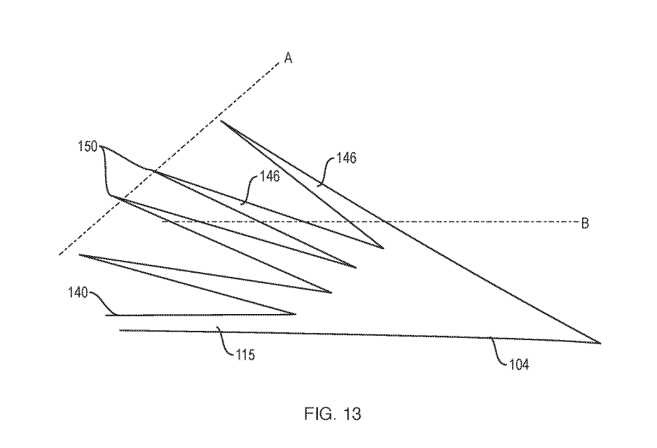

As illustrated in FIG. 13, when the distal end of anchor body 104 is inserted into a bone tunnel (not shown), the free ends 150 of the barbs 146 are moveable from a first position (A) that is further from the top surface 140 of the rib 115 to the second position (B) that closer to the top surface 140 of the rib 114. Compression of the barbs 146 from the first to the second position (A, B) reduces the amount of insertion force required to insert the anchor body 104 into bone. Conversely, when a pull-out force is exerted on the anchor body 104 from a proximal direction, the barbs 146 flex outwardly from the second position (B) towards the first position (A), enhancing the interference fit of the anchor body 104 against the bone and thus increasing pull-out resistance.

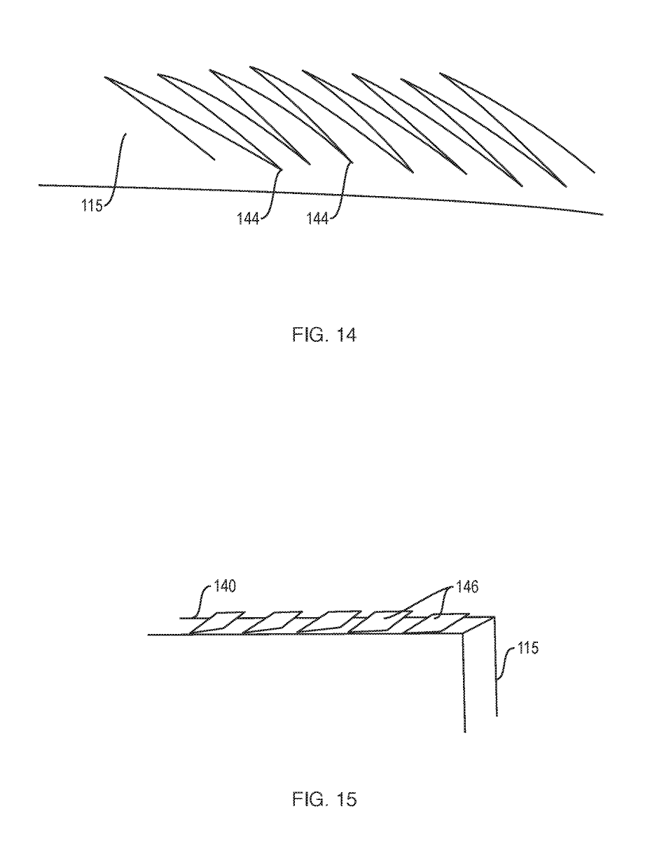

The length of each barb 146 can either be uniform or vary along the length of the rib 115. Where varying lengths occur, the length of the barbs 146 can increase along the length of the rib 115 from the proximal end of the rib 115 to the distal end of the rib 115, or vice versa. The depth to which the cuts 144 encroach into the ribs 115 (compare, for example, FIGS. 11 and 14) may also vary from rib to rib, or could change along the length of the rib 115. It is contemplated by this disclosure that the barbs 146 of one rib 115 may run in a parallel configuration to those of an adjacent rib 115 or the barbs 146 may form a helical or other pattern about the anchor body 104.

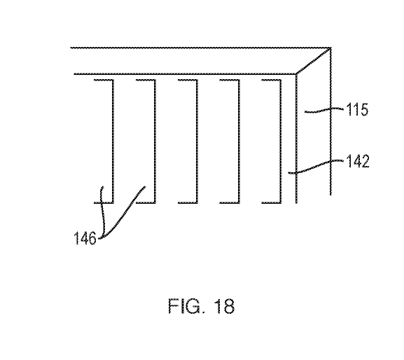

Further examples of the barbs 146 are shown in FIGS. 15-18. In FIG. 15, the barbs 146 are shown as formed in the upper surface 140 of the rib 115. The width of each barb 146 is shown as equal in width to the top surface 140 of rib 115 but other widths are possible. In FIGS. 16 and 17, the barbs 146 are formed instead in the edges of the rib 115, FIG. 17 being a section through the embodiment of FIG. 16. The barbs 146 may also be formed in either or both opposing sidewalls 142 of the ribs 115, as shown in FIG. 18. It is contemplated by this disclosure that the barbs 146 can be formed in one or more surface as shown in FIGS. 15-18 separately or together in one rib 115.

Although the barbs 146 are illustrated in FIGS. 11-18 with reference to the anchor body 104 of FIG. 1, it is contemplated that the barbs 146 may be a feature of any variety of anchor, plug, shank, pin, tack or other surgical fixation device that may employ ribs on its surface to enhance fixation strength.

These and other features and characteristics, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of claims.

What is claimed is:

1. A suture anchor comprising:
an elongated anchor body having a proximal end and a distal end, and a longitudinal axis extending between the proximal and distal ends; and
a plurality of elongate ribs extending longitudinally along at least a portion of the anchor body;
wherein at least one of the plurality of ribs comprises at least one compressible barb.

2. The suture anchor according to claim 1, wherein the at least one of the plurality of ribs comprises a plurality of barbs.

3. The suture anchor according to claim 1, wherein each of the plurality of ribs comprises at least one barb.

4. The suture anchor according to claim 1, wherein each of the plurality of ribs comprises a plurality of barbs.

5. The suture anchor according to claim 1, wherein each of the plurality of ribs comprises a pair of sidewalls, and wherein the at least one barb is located in the sidewalls.

6. The suture anchor according to claim 1, wherein each of the plurality of ribs comprises a pair of sidewalls and a top surface, and wherein the at least one barb is located in the sidewalls, the top surface, or both the sidewalls and the top surface.

7. The suture anchor according to claim 6, wherein the at least one barb is located in an edge of the plurality of ribs, between the sidewalls, or between the sidewalls and the top surface.

8. The suture anchor according to claim 1, wherein the at least one barb comprises a first material selected from the group including poly(lactic-co-glycolic) acid (PLGA), β-Tricalcium phosphate (β-TCP) and calcium sulfate, poly-L-lactic acid-hydroxyapatite (PLLA-HA), poly-D-lactide (PDLA), polyether ether ketone (PEEK) or variants thereof, and bioreabsorbable materials.

9. The suture anchor according to claim 2, wherein a length of each barb varies along a length of each rib upon which each barb is located.

10. The suture anchor according to claim 2, wherein a length of each barb is uniform along a length of each rib upon which each barb is located.

11. The suture anchor according to claim 1, wherein the anchor further comprises a tapered tip formed at the distal end of the anchor body.

12. The suture anchor according to claim 11, wherein the tapered tip comprises a distal tip end.

13. The suture anchor according to claim 12, wherein the distal tip end is formed from a second material that is harder than the first material.

14. The suture anchor according to claim 13, wherein the second material has a hardness within the range between about 40 Shore D to about 85 Shore D.

15. The suture anchor according to claim 13, wherein the second material is selected from the group including stainless steels, titanium, titanium alloys, cobalt-chromium alloys, platinum alloys, and palladium alloys, carbon-reinforced polyether ether ketone (PEEK), and glass-reinforced PEEK.

16. The suture anchor according to claim 12, wherein at least one of the plurality of ribs extends from the proximal end of the anchor body to a position proximal to the distal tip end or to a selection position within the tapered tip.

17. The suture anchor according to claim 1, wherein at least a portion of the plurality of elongate ribs are tapered.

18. The suture anchor according to claim 1, wherein the anchor body further comprises an opening transverse to the longitudinal axis configured for the passage of a suture.

19. The suture anchor according to claim 18, wherein the anchor body further comprises a moveable suture trap housed within the opening and configured to impinge a suture within the opening.

20. A suture anchor assembly comprising:
a suture anchor comprising:
an elongated anchor body having a proximal end and a distal end, and a longitudinal axis extending between the proximal and distal ends; and
a plurality of elongate ribs extending longitudinally along at least a portion of the anchor body;
wherein at least one of the plurality of ribs comprises at least one compressible barb; and
an inserter removably coupled to the suture anchor body.

21. The suture anchor assembly of claim 20, wherein the inserter comprises a slot defined by first and second prongs, the slot in communication with an opening transverse to the longitudinal axis of the anchor body and configured for the passage of a suture, the slot allowing for passage of the suture between the first and second prongs when the inserter is removed from the anchor body.
